(19) United States
(12) Patent Application Publication
(10) Pub. No.: US 2026/0335394 A1
(43) Pub. Date: Oct. 29, 2026

(54) COMPOSITION FOR ALLEVIATING NEUROLOGICAL DISORDERS

(71) Applicant: Veyom BHARDWAJ, New Delhi (IN)

(72) Inventor: Veyom BHARDWAJ, New Delhi (IN)

(21) Appl. No.: 19/424,608

(22) Filed: Dec. 18, 2025

(51) Int. Cl. A61K 36/68; A61K 31/375; A61K 36/81; A61P 25/28

(52) U.S. Cl. CPC A61K 36/68; A61K 31/375; A61K 36/81; A61P 25/28; A61K 2236/33

(57) ABSTRACT

An embodiment relates to a composition, comprising a herbal composition comprising a Bacopa extract and an Ashwagandha extract, wherein a ratio of concentration of the Ashwagandha extract to the Bacopa extract in the composition is in range of about 1:1 to 5:1, and wherein the composition is configured to restore viability of a neural cell, decrease cytotoxicity, and scavenge free radicals.

### TECHNICAL FIELD

This disclosure relates to compositions and methods for treating neural and neurological disorders using the compositions. More particularly, the disclosure relates to a herbal composition to alleviate neurological disorder.

### BACKGROUND

“Neurodegenerative diseases include genetic and sporadic disorders associated with progressive nervous system dysfunction. It has been estimated that one of four Americans will develop a neurodegenerative condition in their lifetimes. Generally, however, the underlying mechanisms causing the conditions are not well understood and few effective treatment options are available for preventing or treating neurodegenerative diseases.” [Source: U.S. Pat. No. 10,709,704B2 titled “Treating neural disease with tyrosine kinase inhibitors” published on Jul. 14, 2020]

“Hundreds of millions of people around the world suffer from neurological disorders or have experienced them intermittently, which has significantly reduced their quality of life. The common treatments for neurological disorders are relatively expensive and may lead to a wide variety of side effects including sleep attacks, gastrointestinal side effects, blood pressure changes, etc. On the other hand, several herbal medications have attracted colossal popularity worldwide in the recent years due to their availability, affordable prices, and few side effects. Aromatic plants, sage (Salvia officinalis), lavender (Lavandula angustifolia), and rosemary (Salvia Rosmarinus) have already shown anxiolytics, anti-inflammatory, antioxidant, and neuroprotective effects. They have also shown potential in treating common neurological disorders, including Alzheimer's disease, Parkinson's disease, migraine, and cognitive disorders.” [Source: Faridzadeh et al. (2022) Neuroprotective Potential of Aromatic Herbs: Rosemary, Sage, and Lavender. Frontiers in neuroscience. https://pubmed.ncbi.nlm.nih.gov/35873824/]

“Studies have shown that EBm promotes free radical scavenger mechanisms and protects cells in prefrontal cortex, hippocampus, and striatum against cytotoxicity and DNA damage implicated in AD. It also reduces lipoxygenase activity reducing lipid peroxidation, increases glutathione peroxidase and chelates iron. Administration of EBm was seen to protect the cholinergic neurons and reduce anticholinesterase activity comparable to donepezil, rivastigmine, and galantamine. It also reduces hippocampal β-amyloid deposition and stress-induced hippocampal damage. The neuroprotective effect of EBm is also due to nitric oxide-mediated cerebral vasodilation. EBm improved the total memory score and maximum improvement was seen in logical memory and paired associate learning in humans and reversed phenytoin-induced memory impairment in experimental model. EBm has not shown any serious clinical, neurological, hematological complications, or vital organs damage in experimental studies” [Source: Chaudhari et al. (2017) Neurocognitive Effect of Nootropic Drug Brahmi (Bacopa monnieri) in Alzheimer's Disease. Ann Neurosci. doi: 10.1159/000475900. Epub 2017 May 12.]

“Ashwagandha on the basis of phyto-pharmacological studies proved potential as anti-inflammatory, anti-oxidant, anti-cancer, anti-microbial, anti-malarial, diuretic, sedative, immumonodulatory and cardioprotective properties” [Source: Zahiruddin et al. (2020) Ashwagandha in brain disorders: A review of recent developments. J Ethnopharmacol. 2020 Jul. 15; 257:112876. doi: 10.1016/j.jep.2020.112876. Epub 2020 Apr. 16.]

There is therefore a need for a synergistic herbal composition that combines two or more neuroprotective plant extracts to provide enhanced therapeutic efficacy for the management of neurological disorders.

### BRIEF SUMMARY

The following paragraphs present a summary to provide a basic understanding of one or more embodiments described herein. This summary is not intended to identify key or critical elements or delineate any scope of the different embodiments and/or any scope of the claims. The sole purpose of the summary is to present some concepts in a simplified form as a prelude to the more detailed description presented herein.

An embodiment relates to a composition, comprising an herbal composition comprising a purified and isolated Bacopa extract and a purified and isolated Ashwagandha extract, wherein a ratio of concentration of the Ashwagandha extract to the Bacopa extract in the composition is in a range to provide synergistic effects resulting in lower cytotoxicity, and higher viability and neuroprotection, and wherein the composition consisting of 25 μg/mL of the Bacopa extract and 25 μg/mL of the Ashwagandha extract has lower cytotoxicity higher viability and protection than the Bacopa extract and the Ashwagandha extract at same doses of 25 μg/mL each, and wherein the composition consisting of 1 mg/ml of the Bacopa extract and 5 mg/ml of the Ashwagandha extract has higher inhibition measured according to 2,2-diphenyl-1-picrylhydrazyl (DPPH) assay than the Bacopa extract and the Ashwagandha extract at same doses of 1 mg/ml and 5 mg/ml respectively.

In an embodiment, the composition yields a % viability value of about 84% as measured according to MTT assay.

In another embodiment, the composition yields a % protection value of about 55% to 65% as measured according to MTT assay.

In yet another embodiment, the composition has an antioxidant effect of about 75% or more as measured using reduction of DPPH radical.

In yet another embodiment, the composition results in a % cytotoxicity value that is at least 2% to 5% lower than obtained with the Bacopa extract and the Ashwagandha extract at same doses of 25 μg/mL each as measured according to MTT assay.

In yet another embodiment, the said composition is configured to treat a neural disorder.

In yet another embodiment, the said composition comprises an active agent.

In yet another embodiment, the active agent is selected from the group consisting of pregnenolone, galangin, vinpocetine, astaxanthin, and huperzine A.

In yet another embodiment, the composition further comprises a vitamin.

In yet another embodiment, wherein the vitamin comprises Vitamin C.

In yet another embodiment, the amount of Vitamin C is at least 100 times less than the Ashwagandha extract and the Bacopa extract.

In yet another embodiment, the ashwagandha extract and/or the Bacopa extract is in an organic solution.

In yet another embodiment, the organic solution is Dimethyl sulphoxide (DMSO).

In yet another embodiment, the composition is configured to treat a neural disorder.

In yet another embodiment, the composition has % DPPH inhibition more than 20 μg/ml of ascorbic acid.

In yet another embodiment, the composition further comprises one or more excipient.

In yet another embodiment, the composition is formulated as an oral dosage form.

In yet another embodiment, the oral dosage form is selected from the group consisting of a tablet, capsule, and caplet.

In yet another embodiment, the one or more excipient selected from the group consisting of calcium carbonate, croscarmellose sodium, dicalcium phosphate, magnesium stearate, microcrystalline cellulose, modified cellulose, silica, and stearic acid.

In yet another embodiment, the composition is configured to increase an enzyme activity level of at least one antioxidant enzyme selected from the group consisting of superoxide dismutase and catalase and decreases the plasma concentration level of reactive chemical species, when administered in an effective amount to a mammalian subject in need thereof.

In yet another embodiment, the increase in enzyme activity level comprises an increase in the activity of at least one antioxidant enzyme selected from the group consisting of superoxide dismutase, catalase, and glutathione peroxidase, the increase being at least about 5%, or being within a range selected from about 5% to about 15%, about 10% to about 30%, about 20% to about 50%, about 30% to about 75%, about 40% to about 100%, about 50% to about 150%, about 75% to about 200%, about 100% to about 300%, about 150% to about 500%, about 200% to about 800%, or about 300% to about 1000%, relative to a corresponding untreated control.

An embodiment relates to a method of treatment of a neural disorder, comprising taking a composition of claim 1, administering the composition to a mammal, and treating the neural disorder.

In an embodiment, the said administering comprises at least one of intravenous administration, intramuscular administration, intranasal administration, intrathecal administration, intrastriatal administration, intracranial administration, intraarterial administration, and subcutaneous administration.

In another embodiment, the administration is in an oral dosage form.

An embodiment relates to a method of treatment, comprising taking a composition, comprising an herbal composition comprising an effective amount of Ashwagandha extract and Bacopa extract in a weight ratio of 1:1 to 4:1, wherein the composition provides synergistic effects resulting in lower cytotoxicity, higher viability and protection, and administering the composition to a mammal, and wherein the composition is configured to reduce % cytotoxicity, increase % viability, and increase % protection more relative to administration of Ashwagandha extract and Bacopa extract alone.

In an embodiment, the composition increases % viability relative to Ashwagandha extract alone.

In another embodiment, the composition increases % viability relative to Bacopa extract alone.

In yet another embodiment, the composition increases % protection relative to either compound alone.

In yet another embodiment, the composition reduces % cytotoxicity relative to either compound alone.

In yet another embodiment, % viability is increased by 2-4% relative to Ashwagandha extract alone.

In yet another embodiment, % viability is increased by 3-5% relative to the Bacopa extract alone.

In yet another embodiment, % protection is increased by 5-8% relative to Ashwagandha extract alone.

In yet another embodiment, % protection is increased by 8-12% relative to Bacopa extract alone.

In yet another embodiment, % cytotoxicity is reduced by 2-5% relative to either compound alone.

In yet another embodiment, Ashwagandha extract is administered at 10-50 μg/mL.

In yet another embodiment, Bacopa extract is administered at 10-50 μg/mL.

In yet another embodiment, Ashwagandha extract and Bacopa extract are each administered at 25 μg/mL.

In yet another embodiment, the composition comprising the Ashwagandha extract and the Bacopa extract at 25 μg/mL each yields a % viability of 84-85%, a % protection of 58-62%, and a % cytotoxicity of 15-17%.

An embodiment relates to a composition, comprising an herbal composition comprising a purified and isolated Bacopa extract and a purified and isolated Ashwagandha extract, wherein a ratio of concentration of the Ashwagandha extract to the Bacopa extract in the composition is in a range of to provide synergistic effects resulting in enhanced antioxidant effect, and wherein the composition consisting of 1 mg/ml of the Bacopa extract and 5 mg/ml of the Ashwagandha extract has enhanced antioxidant effect than the Bacopa extract 1 mg/ml and the Ashwagandha extract at same doses of 5 mg/mL each.

### BRIEF DESCRIPTION OF THE FIGURES

These and other aspects of the present disclosure will now be described in more detail, with reference to the appended drawings showing exemplary embodiments of the present disclosure, in which:

FIG. 1 illustrates a schematic representation of an herbal composition comprising Ashwagandha extract and Bacopa extract.

FIG. 2 illustrates a method of treatment using the herbal composition comprising Ashwagandha and Bacopa extracts, according to one or more embodiments.

FIG. 3 shows antioxidant potential of Ashwagandha and Bacopa as determined based on the ability to scavenge stable radical by DPPH.

FIG. 4 shows for neuroprotection of SHSY-5Y cells against MPP+ iodide induced neuronal damage as evaluated by MTT assay.

Other features of the present embodiments will be apparent from the accompanying drawings and from the detailed description that follows.

### DETAILED DESCRIPTION

A synergistic herbal composition that combines two or more neuroprotective plant extracts capable of offering complementary mechanisms of action, including antioxidant, anti-inflammatory, and neuroregenerative effects, while maintaining a favorable safety and tolerability profile compared to conventional pharmacological therapies. In particular, a synergistic formulation of Ashwagandha (Withania somnifera) and Brahmi (Bacopa monnieri) is desirable to harness their combined potential in restoring neural function, improving cognitive performance, mitigating oxidative stress, and providing sustained neuroprotection in subjects suffering from or at risk of developing neural disorders such as Alzheimer's disease, Parkinson's disease, cognitive impairment, anxiety, and neurodegenerative stress.

Before the present disclosure is described in greater detail, it is to be understood that this invention is not limited to particular embodiments described, as such may, of course, vary. It is also to be understood that the terminology used herein is for the purpose of describing particular embodiment only, and is not intended to be limiting, since the scope of the present invention will be limited only by the appended claims.

It is to be appreciated that the various modes of treatment or prevention of medical conditions as described are intended to mean “substantial”, which includes total but also less than total treatment or prevention, and wherein some biologically or medically relevant result is achieved.

As used herein, the term “neural disorder” refers to any disease, condition, impairment, dysfunction, or pathological state that affects the nerves or the nervous system, or both. Neural disorders include conditions characterized by abnormalities in neuronal survival, synaptic function, neurotransmission, oxidative balance, neuroinflammation, neuronal metabolism, cognition, mood regulation, or motor control. Neural disorders may be acute, chronic, progressive, degenerative, developmental, traumatic, genetic, metabolic, infectious, toxic, or idiopathic in origin.

As used herein, the term “composition” refers to complete set of ingredients that make up a pharmaceutical product. This includes both the active pharmaceutical ingredient (API), which is responsible for its intended therapeutic effect, and inactive components known as excipients, which help with stability of the API, absorption, and delivery to the body.

As used herein, the term “herbal composition” refers to a composition comprising one or more substances of botanical origin, including extracts, concentrates, powders, or purified phytochemical fractions derived from medicinal plants, herbs, or their parts such as roots, stems, leaves, seeds, fruits, or flowers. The herbal composition may be formulated alone or in combination with suitable excipients, carriers, or diluents to provide a therapeutically effective dosage form. The term encompasses both aqueous and organic extracts, whether standardized or non-standardized, and includes compositions wherein the bioactive constituents are derived substantially from plant sources.

An embodiment relates to a composition comprising a herbal composition. The herbal composition comprises Bacopa extract and an Ashwagandha extract.

In an embodiment, the herbal composition consists essentially of a Bacopa extract and an Ashwagandha extract. As used herein, “consisting essentially of” indicates that the composition includes these essential herbal components and may optionally contain additional constituents (e.g., stabilizers, solvents, or excipients) that do not materially affect the essential characteristics or bioactivity of the composition.

In an embodiment, the herbal composition comprises a ratio of concentration of the Ashwagandha extract to the Bacopa extract in range of about 1:1 to 5:1. The concentration ratio of the Ashwagandha extract to the Brahmi extract is in a range of about 1:1 to about 5:1, such that the relative proportion of Ashwagandha extract is equal to or greater than that of the Bacopa extract.

In certain embodiments, the ratio may be about 1.25:1, 1.5:1, 1.75:1, 2:1, 2.5:1, 2.75:1, 3:1, 3.2:1, 3.5:1, 3.75:1, 4:1, 4.5:1 or 4.75:1 providing flexibility to optimize the synergistic antioxidant and neuroprotective properties of the two extracts. In an embodiment, the herbal composition comprises a ratio of concentration of the Ashwagandha extract to the Bacopa extract in range of about 1:5 to 5:1.

Without being limited by theory, the specified ratio ensures a balanced contribution of active phytoconstituents, such as withanolides from Ashwagandha and bacosides from Bacopa, resulting in enhanced free-radical scavenging, mitochondrial protection, and neuronal viability compared with either extract alone.

In some embodiments, the concentration of the extracts is measured in terms of weight of the extract/volume of the solution in which the extract is dissolved.

In certain embodiments, the ratio of Ashwagandha extract to the Brahmi extract may be about 1.5:1, 2:1, 3:1, 4:1, or 5:1, providing a balanced or Ashwagandha-dominant composition. Such ratios have been found to yield enhanced antioxidant and neuroprotective efficacy, wherein the Ashwagandha extract contributes adaptogenic and anti-inflammatory effects, while the Bacopa extract enhances cognitive and antioxidant performance. The synergistic interaction within this ratio range promotes superior free radical scavenging activity compared to either extract alone or compositions outside the specified ratio range.

Over the past few decades, free and bound reactive radicals, highly reactive and thereby destructive molecules, have come to be appreciated increasingly for their importance to human health and disease. A radical, or a free radical, is generally understood as a molecule with one or more unpaired electrons in its outer orbital shell. Many molecular species with bound radicals are monoxides or other oxygen containing compounds, generally referred to as reactive oxygen species (ROS). These highly unstable molecules tend to react rapidly with adjacent molecules, donating, abstracting, or even sharing their outer orbital electron(s). This reaction not only changes the adjacent, target molecule, sometimes in profound and beneficial ways, but it can also damage it, or alternatively the unpaired electron can be passed along to the target, i.e., as in a free radical, generating a second unwanted ROS, which can then go on to react positively or detrimentally with a new target. In fact, much of the high reactivity of ROS is due to their generation of such molecular chain reactions, effectively amplifying their effects many fold. Antioxidants afford protection because they can scavenge ROS and free radicals before they cause damage to the various biological molecules, or prevent oxidative damage from spreading, e.g., by interrupting the radical chain reaction of lipid peroxidation. The reactivity of radicals in the body, and the burden on the body that results in eventual pathological conditions, is known as oxidative stress.

The body's antioxidant defence system includes three important natural antioxidants: superoxide dismutase (SOD), catalase (CAT), and glutathione peroxidase (GPX). Studies conducted have indicated that these antioxidants can work synergistically as the reactions they catalyze are metabolically sequential, beginning first with SOD followed by the actions of CAT and GPX.

SODs are a class of enzymes that catalyze reactions similar to the one below: O₂-+O₂-+2H+→H₂O₂+O₂

The role of SOD in the defense system is to remove superoxide radicals, which are a type of ROS. SOD is found in the body, primarily in three forms: (1) in the cytoplasm as Cu—Zn SOD; (2) in the mitochondrion as Mn-SOD; (3) and in an extracellular environment as CuSOD. Accordingly, the body is dependent upon the presence of zinc, copper, and manganese for the manufacture of SOD. During the removal of superoxide radicals by SOD, both hydroxyl and oxygen radicals are produced, which are catalyzed by CAT and GPX respectively.

Catalase (CAT) is present in the peroxisomes of nearly all aerobic cells and serves to protect the cell from the toxic effects of hydrogen peroxide by catalyzing its decomposition into molecular oxygen and water without the production of free radicals.

The overall reaction is as follows: 2H2O2→2H2O+O2.

The protein exists as a dumbbell-shaped tetramer of four identical subunits (220,000 to 350,000 kDa). Each monomer contains a heme prosthetic group at the catalytic center. CAT monomers from certain species (e.g., cow) also contain one tightly bound NADP per subunit. This NADP may serve to protect the enzyme from oxidation by its H₂O₂ substrate. The body's production of CAT is dependent upon the availability of iron.

Glutathione peroxidases (GPXs) are a large class of diverse enzymes which catalyze the reduction of hydrogen peroxide (H₂O₂), organic hydroperoxides and lipid hydroperoxides to the corresponding alcohol using glutathione (GSH; glutamylcysteinylglycine) as the electron donor (Ursini et al., 1995). The GPX enzymes catalyze the reduction of H₂O₂ to water and organic peroxides (R—O—O—H) to the corresponding stable alcohols (R—O—H) using glutathione (GSH) as a source of reducing equivalents: 2 GSH→ROH+GSSG+H2O

GPXs are important in helping to protect cells against oxidative damage, particularly lipid peroxidation. (Flohé et al., 1976). In mammals, the cytosolic (c) GPX family as well as a family of phospholipid hydroperoxide (PH) GPXs possess a selenocysteine residue at their active site. With the exception of phospholipid-hydroperoxide GPX, a monomer, all of the GPX enzymes are comprised of four identical subunits (monomer Mr 22-23 kDa). Each subunit contains a molecule of selenocysteine in the enzyme active site. The selenocysteine is thought to participate directly in electron donation to the peroxide substrate and become oxidized in the process. The enzyme then uses glutathione as an electron donor to regenerate the reduced form of the selenocysteine (Ursini, 1995). The GPX enzymes accept a wide variety of organic peroxides as substrates. The body's ability to produce GPX is in part dependent upon the adequate supply of selenium, and the supply of glutathione, a tripeptide that the body produces from the amino acids cysteine, glutamic acid, and glycine. Mammals contain plasma (p) GPXs, which are nonselenium containing enzymes in which cysteine replaces selenocysteine at their active site (Ursini et al., 1995). However, with the exception of phospholipid hydroperoxide GPX and perhaps GPX7 or GPX8, the enzymes exhibit a strong preference for glutathione as a source of reducing equivalents.

Despite the presence of SOD, CAT, and GPX, the body's antioxidant defense system is constantly subject to oxidative stress, and its ability to produce SOD, CAT, and GPX is compromised by the aging process and can further be impaired by inflammation, microbial or viral infections, the progression of cancer and neurological disorders, and other pathological conditions that produce, are caused by, or are exacerbated by oxidative stress.

The active agents of the invention are formulated into a composition that retains the prophylactic and therapeutic antioxidant inducing properties of the individual active agents, providing synergistic antioxidant inducing effect relative to the effect of each active agent alone, while also decreasing the toxic side effect(s) to a subject, of the individual active agents of the compositions. The compositions of the invention are useful to eradicate free and bound radical reactions presently taking place or it may be used as prophylaxis against pathological free or bound radical reactions, which may occur as a result of a possible oxidant promoting incident (e.g., ischemic injury).

The composition provides a synergistic combination of herbal extracts which induces enzymes such as SOD, CAT, and GPX and provides the beneficial activities thereof, with pronounced results in neurons. Studies have indicated that the bacosides also increase protein and serotonin levels, while decreasing norepinephrine concentration in the hippocampus, hypothalamus, and cerebral cortex. Bacopa thus reduces the neurodegeneration in the brain that is caused by oxidative stress related to the accumulation of neurotoxic free radicals in the brain.

Accordingly, the composition of the present disclosure may be used to alleviate symptoms of neurodegenerative disorders, such as memory loss, Alzheimer's disease, and Parkinson's disease, and even aging.

Enhancement or in enzyme activity level may include higher catalytic rate (e.g., increased conversion of superoxide radicals to hydrogen peroxide by SOD), higher substrate turnover compared to untreated or control conditions, upregulation of enzyme expression leading to greater total functional enzyme activity, or improved efficiency of endogenous antioxidant pathways. The increase may be relative to a baseline or control, such as untreated cells, placebo, or a reference standard. The enhancement may be measured using biochemical assays, such as spectrophotometric activity assays, kinetic assays, glutathione cycling assays, or ROS-neutralization assays.

In certain embodiments, administration of the composition results in an increase in an enzyme activity level of at least one antioxidant enzyme, such as superoxide dismutase, catalase, or glutathione peroxidase. The increase in enzyme activity may be at least about 5%, or may fall within a range selected from about 5% to about 15%, about 10% to about 30%, about 20% to about 50%, about 30% to about 75%, about 40% to about 100%, about 50% to about 150%, about 75% to about 200%, about 100% to about 300%, about 150% to about 500%, about 200% to about 800%, or about 300% to about 1000%, relative to the activity level measured in a corresponding untreated control sample. The increase may be assessed using any suitable biochemical, spectrophotometric, kinetic, or ROS-neutralization assay known in the art.

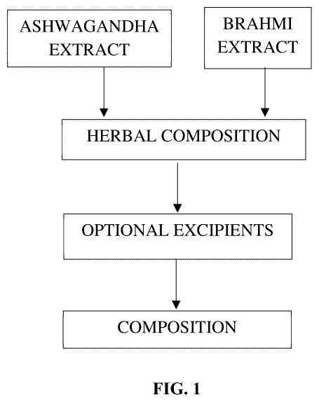

As shown in FIG. 1, the composition includes Ashwagandha extract and Bacopa extract as primary herbal actives, which are combined in a ratio ranging from about 1:1 to about 5:1 (Ashwagandha: Bacopa) to form the herbal composition. The composition may optionally include one or more excipients, which serve as formulation aids, stabilizers, or carriers to support the delivery and stability of the herbal actives. The one or more excipients may also optimize formulation uniformity, tablet or capsule integrity, suspension stability, or powder flowability, thereby ensuring consistent dosing and long-term storage stability.

As used herein, the term “excipient” refers to a pharmaceutically acceptable, non-active substance that is included in a composition to facilitate formulation stability, manufacturability, delivery, or bioavailability of the active agent(s). Excipients may serve one or more functional roles, including as diluents, binders, disintegrants, lubricants, glidants, stabilizers, coating agents, or bulking agents, depending on the intended dosage form and route of administration. Excipients are typically inert and non-reactive with the active ingredients but may influence physicochemical properties such as dissolution rate, flow characteristics, compressibility, and moisture retention.

Excipients and carriers may be same or different in a composition of certain embodiments of the invention.

As used herein, the term “carrier” refers to a non-toxic, inert solid, semi-solid liquid filler, diluent, encapsulating material, formulation auxiliary of any type that aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

The choice of a carrier for use in a composition will depend upon the intended route of administration for the composition.

Suitable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO.

In an embodiment, one or more excipients are selected from the group consisting of calcium carbonate; croscarmellose sodium; dicalcium phosphate; magnesium stearate; microcrystalline cellulose; modified cellulose; silica; and stearic acid.

In some embodiments, the excipient content is from about 1% to about 80% by weight of the total composition, depending on the dosage form and concentration of herbal actives. In liquid formulations, such excipients may further include suspending agents, emulsifiers, solubilizers, or viscosity modifiers such as polysorbates, glycerol, or polyethylene glycols.

In certain embodiments, the excipients are pharmaceutically acceptable according to recognized compendial standards such as USP-NF, EP, or JP, and are compatible with both Ashwagandha and Bacopa extracts, ensuring no adverse impact on their antioxidant or neuroprotective activities.

The composition is configured to restore the viability of neural cells, decrease cytotoxicity, and enhanced antioxidant effect from 2%-81% as determined by reduction potential of 2,2-diphenyl-1-picrylhydrazyl (DPPH) radical by the composition thereby mitigating oxidative stress-induced neuronal damage. In particular, the composition promotes cellular recovery by enhancing mitochondrial function, stabilizing neuronal membranes, and upregulating endogenous antioxidant defenses such as superoxide dismutase (SOD), catalase, and glutathione peroxidase. The Ashwagandha extract contributes to attenuation of neuronal apoptosis and reduction of reactive oxygen species generation, while the Bacopa extract enhances synaptic resilience and prevents lipid peroxidation within neuronal membranes. Through these complementary mechanisms, the composition effectively reduces the cytotoxic effects of neurotoxins such as MPP+, hydrogen peroxide, or glutamate, leading to a measurable increase in cell viability in in vitro assays.

As used herein, the term “viability” or refers to how many cells are “alive” (functional) vs. dead in a sample. In the specific embodiment, cells are neuronal cells.

The term, “increasing viability”, or “restoring viability” or “higher viability” refers to increase in biological activity of the cells such as a metabolic activity, membrane integrity, or ATP content in the cells following exposure to an injurious stimulus, so that the number of dead cells decreases. The viability is measured by a MTT viability assay.

In certain embodiments, restoring viability comprises increasing neural cell viability by about 10% to about 80%, for example by about 10% to about 25%, about 25% to about 50%, or about 50% to about 80%, relative to an injured control.

The term “decrease cytotoxicity” refers to a reduction in cellular damage or death, as measured according MTT assay. The decrease in cytotoxicity may occur due to at least one of mechanism including reductions in LDH release, propidium iodide uptake, apoptotic markers, or necrotic membrane disruption. In certain embodiments, decreasing cytotoxicity comprises reducing cytotoxicity by about 10% to about 50%, with the minimum range selected from 10%, 12%, 15% and the maximum range selected from 15, 20, 30 or more. In certain embodiments, decrease in cytotoxicity is about 15% to about 30%, about 30% to about 50%, or about 50% to about 70%, relative to an untreated injured control.

The term “scavenge free radicals” refers to decreasing or neutralizing intracellular reactive oxygen species (ROS), superoxide, or other oxidizing species, as quantified by DPPH assays. In certain embodiments, scavenging free radicals comprises reducing intracellular ROS levels by about 20% to about 85%, for example by about 20% to about 40%, about 40% to about 60%, or about 60% to about 85%, compared to an injured or oxidatively stressed control, wherein combination treatments can achieve inhibition in the higher range (e.g., 75% at 5 mg/ml Ashwagandha plus 1 mg/ml Brahmi) compared to corresponding single treatments (e.g., 57% at 5 mg/ml Ashwagandha or 53% at 1 mg/ml Brahmi).

In certain embodiments, the composition demonstrates at least a 50% restoration of neuronal viability relative to untreated controls following exposure to oxidative or neurotoxic stress, while concurrently exhibiting a significant reduction in intracellular ROS levels and free radical scavenging activity exceeding 70% in standard assays such as DPPH assay. In particular, the composition comprising both Ashwagandha (5 mg/ml) and Brahmi extract (1 mg/ml) achieves about 75% inhibition, exceeding the corresponding individual activities at the same concentrations, thereby demonstrating a synergistic effect. In certain embodiments, the composition demonstrates at least a 55%, 60%, 65%, 70%, 75%, 80%, 85%, 90% or more restoration of neuronal viability relative to untreated controls following exposure to oxidative or neurotoxic stress.

In an embodiment, the composition is configured to restore viability of a neural cell, decrease cytotoxicity, and scavenge free radicals. Collectively, these properties indicate that the composition exerts a synergistic neuroprotective and antioxidative effect suitable for therapeutic use in managing neurodegenerative or oxidative stress-related disorders.

As used herein, the term “protection” refers to the prevention, reduction, mitigation, delay, or attenuation of biological damage or functional impairment in a cell, tissue, organ, or organism. Protection includes any measurable improvement in structural integrity, physiological function, biochemical stability, or resistance to harmful stimuli when compared to an untreated or negative control. In certain embodiments, protection comprises decreasing cytotoxicity, reducing oxidative stress, limiting inflammatory responses, preserving mitochondrial function, maintaining membrane integrity, inhibiting apoptotic pathways, or preventing biomolecular degradation. The term encompasses both partial and complete protection and includes statistically significant improvements in any relevant protective parameter over a defined period.

As used herein, the term “neuroprotection” refers to the prevention, reduction, attenuation, or delay of neural damage, dysfunction, or loss in the central or peripheral nervous system. Neuroprotection includes maintaining neuronal viability, preserving synaptic integrity, limiting apoptosis or necrosis, reducing oxidative injury or reactive oxygen species, stabilizing mitochondrial function, decreasing neuroinflammation, preventing excitotoxicity, and maintaining normal neurotransmission or neurochemical balance. The term further encompasses enhancing endogenous defense pathways, promoting recovery of neural cells following injury, preventing progressive neurodegeneration, and improving overall structural or functional outcomes relative to untreated neural cells or subjects. Neuroprotection may be acute or sustained and is demonstrated by measurable improvements in neuronal survival, function, morphology, or biochemical markers of neural health.

In an embodiment, Ashwagandha extract is present at a concentration of about 5 mg/mL, and the Bacopa extract is present at a concentration of about 1 mg/mL. The resulting Ashwagandha-to-Bacopa concentration ratio of approximately 5:1 provides a synergistic balance between adaptogenic, neuroprotective, and antioxidant activities.

In certain embodiment, in a composition, Ashwagandha extract could be either about 25 μg/ml, 50 μg/ml, 100 μg/ml, 1000 μg/ml, 1 mg/ml, 2 mg/ml, 3 mg/ml, 5 mg/ml, 7.5 mg/ml, 10 mg/ml or more, and the Bacopa extract could be either 25 μg/ml, 50 μg/ml, 100 μg/ml, 1000 μg/ml, 1 mg/ml, 2 mg/ml, 3 mg/ml, 5 mg/ml, 7.5 mg/ml, 10 mg/ml or more.

In certain embodiments, the concentration of the Ashwagandha extract may range from about 4 mg/mL to about 6 mg/mL, and the concentration of the Bacopa extract may range from about 0.8 mg/mL to about 1.2 mg/mL, maintaining an overall Ashwagandha-to-Bacopa ratio of about 4:1 to about 6:1. Concentrations within this range have been found to provide consistent and reproducible antioxidant performance while preserving formulation stability and bioactivity during storage and use.

The composition at the mentioned concentration ratio in one of the embodiments contributes significantly to the reduction of oxidative stress and inflammation through modulation of reactive oxygen species and enhancement of endogenous antioxidant enzyme activity.

In an embodiment, the ashwagandha extract and/or the Bacopa extract is in an organic solution. The Ashwagandha extract and/or the Bacopa extract is formulated or dissolved in an organic solution to facilitate solubilization of bioactive phytoconstituents, enhance extract stability, and enable uniform dispersion within the composition. The organic solution provides a medium suitable for preserving the antioxidant and neuroprotective integrity of the extracts during processing and storage.

Non-limiting examples of suitable organic solvents include alcohols such as ethanol, methanol, isopropanol, and butanol; esters such as ethyl acetate and propylene glycol diacetate; ketones such as acetone or methyl ethyl ketone; and polar aprotic solvents such as dimethyl sulfoxide (DMSO), dimethylformamide (DMF), N-methyl-2-pyrrolidone (NMP), or ethyl lactate. In certain embodiments, biocompatible or pharmaceutically acceptable organic solvents such as ethanol, propylene glycol, glycerol, polyethylene glycol (PEG-400), triacetin, or benzyl alcohol are preferred for preparing dosage forms intended for oral, injectable, or implantable administration.

The concentration of the extract in the organic solution may vary depending on solvent polarity, solubility characteristics of the active components, and intended route of administration. In some embodiments, the concentration of Ashwagandha extract in the organic phase is from about 0.1 mg/mL to about 50 mg/mL, and the concentration of Bacopa extract is from about 0.05 mg/mL to about 20 mg/mL. The use of such organic solutions enhances extract penetration, ensures homogeneity of the formulation, and supports controlled release or delivery when incorporated into pharmaceutical, nutraceutical, or implantable compositions.

In an embodiment, the herbal composition may further include extracts of other herbs, such as Gotu kola (Centella asiatica), and Gingko (Gingko biloba), Ginger (Zingiber officinale), Holy Basil (Ocimum sanctum), Hu Zhang (Polygonum cuspidatum), Oregano (Origanum vulgare, Origanum onites), Rosemary (Rosmarinus officinalis, Rosmarinus eriocalyx, species in the genus Rosmarinus), Turmeric (Curcuma longa), Green Tea (Camellia sinensis), lavender (Lavandula spica and related species in the genus Lavandula), skullcap (Scutellaria lateriflora), Milk Thistle extract, Aloe vera powder, Polyphenols, polyphenol (−)-epigallocatechin gallate (EGCG), Ginko biloba leaf extract and N-Acetyl Cysteine (NAC), and oat straw (Avena sativa, Avena byzantina). Moreover, Salvia divinorum, aka Diviner's Sage, ayahuasca, a concoction made from Banisteriopsis caapi and Psychotria species, and plants containing ibogaine (Tabernanthe iboga, Voacanga Africana and Tabernaemontana undulate), peyote (Lophophora williamsii), the seeds of morning glory (Ipomoea tricolor and related species) and Hawaiian baby wood rose (Argyreia nervosa), and Cannabis (Cannabis sativa, C. indica and C. ruderalis), can be incorporated as well. Any nervine agents from natural products may also be incorporated, including, for example, cordycepin (or Cordyceps extracts containing such) or amyloban (found in Lion's mane).

In an embodiment, the composition exhibits an antioxidant effect of about 75% or greater, as determined by free radical scavenging assays. In one example, the antioxidant potential is measured by reduction of the 2,2-diphenyl-1-picrylhydrazyl (DPPH) radical, wherein the percentage inhibition of DPPH absorbance at 517 nm indicates the radical scavenging capacity of the composition. The high percent inhibition, typically equal to or greater than 75%, reflects the ability of the formulation to neutralize reactive oxygen species and prevent oxidative damage to biomolecules such as proteins, lipids, and nucleic acids.

As used herein, the term “antioxidant effect” refers to the ability of a compound, composition, or cellular treatment to reduce, neutralize, or prevent the formation of reactive oxygen species (ROS), reactive nitrogen species (RNS), or other chemically reactive free radicals within a biological system. The antioxidant effect may include one or more of: (i) scavenging existing free radicals; (ii) inhibiting the generation of new ROS or RNS; (iii) chelating metal ions that catalyze oxidative reactions; (iv) enhancing endogenous antioxidant pathways, such as glutathione, catalase, or superoxide dismutase activity; and (v) preventing oxidative damage to biomolecules including lipids, proteins, nucleic acids, and cellular membranes. The antioxidant effect may be quantified using assays such as DCF-DA ROS measurements, lipid peroxidation assays (e.g., MDA, TBARS), ORAC assays, or antioxidant enzyme activity assays.

Non-limiting examples of suitable analytical assays for quantifying the antioxidant effect comprise the DPPH radical scavenging assay, the ABTS [2,2′-azino-bis(3-ethylbenzothiazoline-6-sulfonic acid)] cation radical decolorization assay, the Ferric Reducing Antioxidant Power (FRAP) assay, the Oxygen Radical Absorbance Capacity (ORAC) assay, the Hydroxyl Radical Scavenging (HRSA) assay, the Superoxide Anion Scavenging assay, and the Nitric Oxide Radical Inhibition assay.

In certain embodiments, the antioxidant activity of the composition is comparable to or greater than that of a reference standard such as ascorbic acid (vitamin C), trolox, or butylated hydroxytoluene (BHT), when evaluated under equivalent experimental conditions. The observed antioxidant activity of about 75% or more indicates that the composition effectively scavenges free radicals, thereby mitigating oxidative stress and contributing to cellular protection and neurorestorative effects.

In certain embodiments, the composition restores cellular viability to a level ranging from 105% to 150%, preferably 110% to 140%, and more preferably 115% to 130% relative to untreated control cells. In another embodiment, the composition provides neuroprotection such that viability under stress conditions is maintained within 90% to 120%, preferably 95% to 115%, and most preferably 100% to 110% of the control level. In yet another embodiment, the composition demonstrates dose-dependent effects, wherein lower concentrations achieve viability restoration of 100% to 110%, while higher concentrations achieve enhancement up to 130% or greater, thereby confirming both protective and restorative properties.

In an embodiment, the composition is configured to restore cellular viability and provide neuroprotection at levels exceeding 100% relative to untreated control cells, as determined using a 3-(4,5-dimethylthiazol-2-yl)-2,5-diphenyltetrazolium bromide (MTT) assay. The MTT assay evaluates mitochondrial metabolic activity as an indicator of viable, metabolically active cells. In one example, neuronal cells exposed to oxidative stress or cytotoxic stimuli demonstrate reduced viability, whereas treatment with the composition restores or enhances viability to more than 100% of the control level, indicating both cytoprotective and neurorestorative effects.

The observed increase in viability suggests that the composition supports cellular recovery, membrane integrity, and mitochondrial function, thereby providing significant neuroprotection against oxidative or toxic insults. Without being bound by theory, such neuroprotective effects may result from the synergistic antioxidant and adaptogenic activities of the Ashwagandha and Bacopa extracts that enhance cellular resilience under stress conditions.

Non-limiting examples of suitable assays for evaluating cell viability and neuroprotection comprise MTT assay (based on mitochondrial reduction of tetrazolium dye), CellTiter-Glo® luminescent cell viability assay (ATP quantification), Trypan blue exclusion assay (membrane integrity), Lactate dehydrogenase (LDH) release assay (cytotoxicity index), Neutral red uptake assay (lysosomal activity), Resazurin (Alamar Blue) reduction assay, Calcein-AM/propidium iodide dual staining assay (live/dead discrimination), and Annexin V/PI flow cytometry assay (apoptosis detection).

In certain embodiments, the composition demonstrates a statistically significant improvement in neuronal survival, metabolic function, and membrane stability relative to untreated or vehicle-treated controls, confirming its neuroprotective efficacy and ability to promote cellular regeneration following oxidative or chemical stress.

In an embodiment, composition is configured to treat a neural disorder. The composition is configured to protect neuronal cells from oxidative or inflammatory damage, restore cellular viability, enhance synaptic transmission, or support neurogenesis and neural repair. The therapeutic configuration may be achieved through one or more biochemical mechanisms, including but not limited to reduction of oxidative stress by scavenging reactive oxygen species (ROS) and inhibiting lipid peroxidation, modulation of neurotransmitter pathways, such as acetylcholine, dopamine, serotonin, and GABAergic systems, inhibition of pro-inflammatory cytokines or microglial activation within the neural environment, regulation of mitochondrial integrity and ATP production, thereby improving neuronal energy homeostasis, enhancement of neurotrophic signaling, such as upregulation of BDNF (brain-derived neurotrophic factor) and NGF (nerve growth factor), and prevention of neuronal apoptosis or synaptic loss under conditions of stress, toxin exposure, or ischemia. Non-limiting examples of such neural disorders include Neurodegenerative disorders (such as Alzheimer's disease, Parkinson's disease, Huntington's disease, amyotrophic lateral sclerosis (ALS), and multiple sclerosis (MS)), Cognitive and memory-related disorders (such as age-related cognitive decline, mild cognitive impairment, attention deficit disorders, and learning disabilities), Neuropsychiatric conditions (such as depression, anxiety, stress-induced cognitive dysfunction, and post-traumatic stress disorder (PTSD), Ischemic or oxidative neural injuries (such as cerebral ischemia, stroke, traumatic brain injury (TBI), and hypoxic-ischemic encephalopathy), and Peripheral nervous system disorders (such as diabetic neuropathy, neuroinflammation, and toxin-induced neuropathies).

In certain embodiments, administration of the composition results in measurable improvement in neuronal viability, reduction in oxidative biomarkers, restoration of neurotransmitter homeostasis, and enhancement of cognitive or motor performance as compared to baseline or untreated conditions.

The composition may further serve as an adjunct or complementary therapy alongside standard neuroprotective, anti-inflammatory, or antioxidant treatments to enhance overall therapeutic efficacy and reduce neurodegenerative progression.

In an embodiment, the composition further comprises an active agent.

As used herein, the term “active agent” refers to any pharmaceutically, nutraceutically, or biologically active compound that, when administered alone or in combination with other components of the composition, elicits a therapeutic, preventive, or restorative effect in a subject. The active agent may act through one or more mechanisms, including but not limited to antioxidant, neuroprotective, adaptogenic, anti-inflammatory, nootropic, cholinergic, or mitochondrial-modulatory pathways.

In an embodiment, the composition comprises an active agent selected from the group consisting of pregnenolone, galangin, vinpocetine, astaxanthin, and huperzine A. These active agents are known to exhibit neuroprotective or cognitive-enhancing properties, including modulation of neurotransmitter release, inhibition of acetylcholinesterase, enhancement of mitochondrial function, and attenuation of oxidative stress or neuroinflammation.

In certain embodiments, the composition may optionally include one or more additional active agents selected from the group consisting of neuroprotective agents, nootropic and cognition-enhancing agents, adaptogenic and anti-stress agents, antioxidant, mitochondrial and metabolic enhancers, neurotrophic and synaptic modulators, and cholinergic and dopaminergic modulators.

Non limiting examples of neuroprotective agents are resveratrol, curcumin, quercetin, luteolin, apigenin, pterostilbene, and coenzyme Q10. Non limiting examples of nootropic and cognition-enhancing agents are piracetam, aniracetam, oxiracetam, citicoline (CDP-choline), alpha-GPC, and L-theanine. Non limiting examples of adaptogenic and anti-stress agents and Panax ginseng extract, Rhodiola rosea extract, Schisandra chinensis extract, and Eleutherococcus senticosus extract. Non limiting examples of antioxidants are tocopherols (vitamin E), ascorbic acid (vitamin C), beta-carotene, selenium compounds, and N-acetylcysteine (NAC). Non limiting examples of mitochondrial and metabolic enhancers are nicotinamide riboside, lipoic acid, acetyl-L-carnitine, and creatine. Non limiting examples of neurotrophic and synaptic modulators are brain-derived neurotrophic factor (BDNF) mimetics, nerve growth factor (NGF) inducers, and phosphatidylserine. Non limiting examples of cholinergic and dopaminergic modulators are L-dopa, selegiline, rasagiline, and cholinesterase inhibitors such as donepezil and galantamine.

In various embodiments, the active agent is present in a therapeutically effective amount to achieve the desired biological response, either alone or in synergistic combination with Ashwagandha extract and/or Bacopa extract. The ratio of the active agent to the botanical extract may be adjusted to optimize bioavailability, cellular uptake, and neuroprotective efficacy.

Without being bound by theory, inclusion of the active agent enhances the multimodal neuroprotective profile of the composition, providing both acute antioxidant activity and long-term neuroregenerative benefits.

In an embodiment, the composition further comprises a vitamin, which may function as a co-antioxidant, stabilizing agent, or bioactivity enhancer. The inclusion of a vitamin component can improve the overall antioxidant capacity, maintain redox balance within neuronal cells, and support the synergistic neuroprotective effects of the Ashwagandha and Bacopa extracts.

In another embodiment, the vitamin comprises Vitamin C (ascorbic acid). Vitamin C acts as a water-soluble antioxidant that scavenges reactive oxygen species (ROS), regenerates other antioxidants such as vitamin E, and contributes to collagen synthesis and cellular repair. The presence of Vitamin C also enhances bioavailability and metabolic activation of plant-derived phytoconstituents such as anolides and bacosides.

In certain embodiments, the amount of Vitamin C is at least 100 times less, and in some cases between 50 to 500 times less than the total amount of Ashwagandha extract and Bacopa extract combined, ensuring that the vitamin functions primarily as a supportive antioxidant rather than serving as the primary active component. In other embodiments, the ratio may range from about 1:100 to about 1:1000, depending on the desired formulation balance and intended therapeutic effect. For example, where the Ashwagandha extract is about 5 mg/mL and the Bacopa extract is about 1 mg/mL, the Vitamin C may be present at a concentration of 0.01 mg/mL or less.

In various embodiments, the concentration of Vitamin C may be in a range of about 0.0001 mg/mL to about 0.5 mg/mL, or about 0.001 wt % to about 0.05 wt % of the total composition, depending on formulation type and delivery route.

In some embodiments, the vitamin component may include one or more additional vitamins, such as retinol, beta-carotene, tocopherol, cholecalciferol, Vitamin B complex (including B1, B2, B3, B5, B6, B9, and B12, and menaquinone.

In certain embodiments, the combination of Vitamin C with Ashwagandha and Bacopa extracts results in synergistic antioxidant and neuroprotective effects, reducing oxidative stress-induced cytotoxicity and promoting neuronal survival beyond levels observed with individual components alone. Without being bound by theory, such synergy may arise from redox cycling between ascorbate and phenolic constituents of the extracts, facilitating prolonged antioxidant activity within neural tissues.

In an embodiment, the herbal composition shows % DPPH inhibition comparable to ascorbic acid at concentrations greater than 20 μg/ml, more preferably between 25-75 μg/ml, and most preferably around 50 μg/ml. As used herein, the term “inhibiting” refers to reducing, suppressing, decreasing, limiting, preventing, or slowing the progression, activity, occurrence, or severity of free radicals. “Inhibiting” does not require complete elimination; rather, any detectable, measurable, or statistically significant reduction is encompassed within the meaning of the term. Inhibition may occur partially or fully, may be direct or indirect, and may result from one or more mechanisms including, but not limited to, antioxidant action, receptor modulation, enzyme regulation, or mitigation of cellular stress.

In an embodiment, the herbal composition exhibits an antioxidant activity corresponding to a percentage inhibition of inhibition greater than that observed for 20 μg/mL of ascorbic acid when measured using a free radical scavenging assay such as the 2,2-diphenyl-1-picrylhydrazyl (DPPH) method. This result indicates that the composition possesses superior radical-quenching potential compared to the reference antioxidant standard, ascorbic acid, under equivalent test conditions.

Without being bound by theory, the enhanced inhibitory effect is attributed to the synergistic action of phytoconstituents present in the Ashwagandha and Bacopa extracts, such as but not limited to with anolides, bacosides, flavonoids, and polyphenolic compounds, which collectively contribute to hydrogen- or electron-donating capacity, thereby neutralizing reactive oxygen species (ROS) and stabilizing free radicals.

In certain embodiments, the herbal composition achieves greater than 75% inhibition of DPPH radicals, exceeding the antioxidant efficiency of 20 μg/mL ascorbic acid by at least 20%, thereby confirming its potent free-radical scavenging and cytoprotective properties suitable for mitigating oxidative stress-related neural damage.

In an embodiment, the composition is configured to increase the activity level of one or more endogenous antioxidant enzymes when administered in an effective amount to a mammalian subject in need thereof. The at least one antioxidant enzyme is selected from the group consisting of superoxide dismutase (SOD) and catalase (CAT).

Without being bound by theory, administration of the composition stimulates or up-regulates the endogenous antioxidant defense system, leading to enhanced enzymatic degradation of reactive oxygen species (ROS) and reduction of oxidative stress within neural and peripheral tissues. The upregulation of superoxide dismutase facilitates the dismutation of the superoxide anion radical (O₂·) into hydrogen peroxide (H₂O₂), while catalase promotes the rapid conversion of hydrogen peroxide into water and molecular oxygen, thereby minimizing oxidative injury to lipids, proteins, and DNA.

In certain embodiments, administration of the composition results in a statistically significant increase in SOD and/or CAT activity levels, for example by about 10% to about 200%, relative to baseline or untreated controls, as determined by spectrophotometric or fluorometric enzyme activity assays.

Concomitantly, the composition is configured to decrease the plasma concentration levels of reactive chemical species, including but not limited to reactive oxygen species (ROS), reactive nitrogen species (RNS), malondialdehyde (MDA), nitric oxide (NO), and hydrogen peroxide (H₂O₂). This decrease reflects attenuation of lipid peroxidation and oxidative damage in vivo. In various embodiments, administration of the composition results in a reduction of such reactive species by at least 10% to 75%, depending on dosage and treatment duration.

In particular embodiments, the Ashwagandha and Bacopa extracts act synergistically to activate cellular antioxidant pathways such as the Nrf2-Keap1 signaling cascade, thereby promoting transcriptional upregulation of antioxidant response element (ARE)-driven genes, including SOD, CAT, and glutathione peroxidase (GPx).

In some embodiments, enhancement of antioxidant enzyme activity and reduction of reactive chemical species collectively lead to restoration of redox homeostasis, protection of neuronal cells, and improvement of physiological or behavioral outcomes in models of oxidative or neurodegenerative disorders.

In an embodiment, the composition can also contain adjuvant. Adjuvants are not active agents. “Adjuvant” or “stimulant” or “immunostimulating agents” or similar refers to a compound that enhances a subject's immune response to an antigen when administered in conjunction with the antigen. Adjuvants or immunostimulating agents usually act via their capability to induce an innate immune response.

### Dosage Formulation

As used herein, the term “dosage form” refers to the physical form in which a pharmaceutical or nutraceutical composition is produced, stored, and administered to a subject. The dosage form determines the route of administration, release characteristics, stability profile, and bioavailability of the active ingredients. Examples of dosage forms include, but are not limited to, tablets, capsules, powders, granules, suspensions, emulsions, syrups, solutions, gels, and sustained-release systems. The selection of a dosage form is guided by factors such as intended therapeutic use, desired pharmacokinetic profile, stability of the actives, and patient compliance.

In an embodiment, the composition is formulated as an oral dosage form. Oral dosage forms are particularly advantageous for ease of administration, patient compliance, and suitability for chronic or prophylactic treatment of neural disorders. The oral dosage form may be configured to deliver a therapeutically effective amount of the active ingredients-such as Ashwagandha extract and Bacopa extract, while maintaining their stability and bioactivity during gastrointestinal transit and systemic absorption.

In various embodiments, the oral dosage form may be selected from, but is not limited to, the tablets, including compressed tablets, chewable tablets, or effervescent tablets, capsules, including hard gelatin capsules, hydroxypropyl methylcellulose (HPMC) capsules, or soft gelatin capsules, granules or powders intended for direct ingestion or reconstitution in a liquid vehicle, oral liquids, such as solutions, suspensions, or emulsions containing pharmaceutically acceptable carriers, and controlled-release or sustained-release formulations, such as matrix tablets, coated granules, or multiparticulate systems designed to provide prolonged or targeted delivery of active ingredients.

In some embodiments, the oral dosage form further comprises one or more pharmaceutically acceptable excipients, including binders, disintegrants, lubricants, fillers, stabilizers, and flavoring agents, as described elsewhere herein.

In certain embodiments, the oral dosage form may be configured to provide immediate, delayed, or extended release of the active ingredients to achieve optimal plasma concentrations over time. For example, a sustained-release capsule may release Ashwagandha and Bacopa extracts over 8-12 hours to maintain consistent neuroprotective activity.

A wide variety of materials may be used to prepare sustained-release particles, including biodegradable, non-biodegradable, natural, and lipid-based polymers, as well as suitable stabilizers and processing agents. Exemplary biodegradable polymers include poly(lactic acid) (PLA), poly(glycolic acid) (PGA), poly(lactic-co-glycolic acid) (PLGA), polycaprolactone (PCL), polyanhydrides, polyorthoesters, polycarbonates, poly(alkyl cyanoacrylates), poly(amino acids), and polyhydroxyalkanoates. Natural polymers that may be employed include alginate, chitosan, gelatin, collagen, hyaluronic acid, pectin, pullulan, starch derivatives, and cellulose derivatives such as ethylcellulose. Non-biodegradable polymers suitable for sustained release include poly(methacrylate) polymers (e.g., Eudragit® RS, RL, NE, L, and S), polyvinyl alcohol, polyethylene-vinyl acetate, high-molecular-weight polyethylene glycol, and polystyrene. Lipid or wax-based matrix materials may also be utilized and include glyceryl behenate, glyceryl palmitostearate, stearic acid, beeswax, carnauba wax, cholesterol, and phospholipids such as lecithin. Surfactants and stabilizers such as polyvinyl alcohol, poloxamers, Tween® 20, Tween® 80, Span® 20, Span® 60, sodium dodecyl sulfate, lecithin, and carbomers may be incorporated to facilitate particle formation and stability. Cross-linking agents including glutaraldehyde, genipin, calcium chloride, and tripolyphosphate may be used to harden polymer matrices. Osmotic or release-modifying agents such as sodium chloride, potassium chloride, mannitol, sorbitol, lactose, citric acid, and tartaric acid may further modulate hydration and drug-release kinetics. Additionally, plasticizers such as triethyl citrate, diethyl phthalate, glycerin, and propylene glycol may be included to improve polymer flexibility and coating performance. Organic solvents used in the fabrication of sustained-release particles include dichloromethane, ethyl acetate, acetone, ethanol, isopropanol, and dimethyl sulfoxide.

In some embodiments, the oral dosage form is manufactured by direct compression, wet granulation, dry granulation, or encapsulation techniques, using conventional pharmaceutical processes that preserve the integrity of phytoconstituents such as withanolides and bacosides.

In particular embodiments, the oral dosage form is intended for once- or twice-daily administration to deliver a total daily dose equivalent to about 50 mg to 2000 mg of herbal extract composition, optionally in combination with vitamins, minerals, or excipients as described herein.

It is advantageous to formulate oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the dietary supplement and the particular therapeutic effect to be achieved, and the limitations inherent in the art of compounding such an active compound for the treatment of individuals. The compositions can be included in a container, pack, or dispenser together with instructions for administration. Typically, an oral dose is taken two-times to four-times daily, until symptom relief is apparent. The compositions of the present invention can also be administered in combination with each other, or with one or more additional therapeutic compounds.

Solid dosage forms for oral administration of the compounds described herein include capsules, tablets, pills, powders, and granules. In such solid dosage forms, the compounds described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms can also comprise buffering agents.

Solid compositions of a similar type can also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They can contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or pharmaceutically acceptable salts or prodrugs thereof include pharmaceutically acceptable emulsions, solutions, suspensions, syrups, and elixirs. In addition to the active compounds, the liquid dosage forms can contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, can contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

In further embodiments, the Ashwagandha extract and the Bacopa extract may be prepared from Withania somnifera and Bacopa monnieri, respectively, using conventional extraction techniques such as maceration, percolation, Soxhlet extraction, or solvent evaporation. Suitable extraction solvents may include ethanol, methanol, water, hydroalcoholic mixtures, ethyl acetate, or other pharmaceutically acceptable solvents.

The process for preparing the herbal composition comprising Ashwagandha and Bacopa extracts. It begins with the selection and extraction of herbal materials, where Ashwagandha and Bacopa are separately processed using a suitable extraction solvent such as ethanol or water to yield concentrated extracts. The next step involves determining the ratio of Ashwagandha to Bacopa, typically within the range of 1:1 to 5:1 based on desired pharmacological potency and antioxidant activity. The extracts are then combined and homogenized in an appropriate organic or aqueous medium to ensure uniform distribution. Subsequently, optional excipients such as microcrystalline cellulose, magnesium stearate, or silica may be incorporated to improve flowability, compressibility, or stability of the formulation.

Following this, the formulation process diverges into dosage form preparation, such as granulation, drying, and compression (for tablets) or encapsulation (for capsules). The process concludes with quality control and packaging, including standardization for phytochemical content, antioxidant efficacy (e.g., DPPH assay), and stability evaluation.

### Method of Treatment

An embodiment relates to a method of treatment of a neural disorder. The method comprises taking an effective amount of the composition as described in one or more embodiments of present disclosure, comprising a Bacopa extract and an Ashwagandha extract; and administering the composition to a subject to treat the disease.

As used throughout, term, “subject” “subject” as used herein, is preferably a mammal, such as a human, but can also be an animal, e.g., domestic animals (e.g., dogs, cats and the like), farm animals (e.g., cows, sheep, pigs, horses and the like) and laboratory animals (e.g., rats, mice, guinea pigs and the like). Thus, veterinary uses and medical formulations are contemplated herein. A

An “effective amount” of the composition, as used herein, is a quantity of the composition provided by a particular route of administration and at a particular dosing regimen, that is sufficient to achieve a desired therapeutic and/or prophylactic effect. In an embodiment, the “effective amount” or “therapeutic amount” refers to an amount sufficient to have neurogenerative activity. This amount may vary to some degree depending on the mode of administration but will be in the same general range. The exact effective amount necessary could vary from subject to subject, depending on the compound, preventative treatment or condition being treated, the mode of administration, etc. The appropriate effective amount may be determined by one of ordinary skill in the art using only routine experimentation or prior knowledge in the art in view of the present disclosure. Note that effective ranges/dosages are not expected to be precisely the same for all compounds. Dosages may be optimized with each compound when the pharmacokinetics are studied to see how each compound is metabolized, which may alter the dose ranges. Nevertheless, given these factors, this neuro-enhancing invention is best managed in consultation with a skilled medical professional. For example, an amount that results in the prevention of or a decrease in the symptoms associated with a disease that is being treated. The amount of the composition administered to the subject will depend on the type and severity of the disease, the amenability of the disorder to respond to antioxidants, and on the characteristics of the individual and their metabolic ability to respond to the compositions to produce in vivo, the antioxidants, such factors including general health, age, sex, body weight and tolerance to the active agents in the compositions. The skilled artisan will be able to determine appropriate dosages depending on these and other factors. Typically, an effective amount of the compositions of the present invention, sufficient for achieving a therapeutic or prophylactic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. The compositions of the present invention can also be administered in combination with each other, or with one or more additional therapeutic or prophylactic compounds.

As referred herein, “IC50” or “half maximal inhibitory concentration” is a quantitative measure used in pharmacology, biochemistry, and drug development to indicate how much of a particular substance (such as a drug, inhibitor, a composition or compound) is needed to inhibit a biological process or the activity of a target (like an enzyme, receptor, or cell) by 50%. For antioxidant activity of a composition, the IC₅₀ would be the concentration at which the formulation reduces free radical activity by 50%.

In an embodiment, the composition of the invention is a dietary supplement composition. In another embodiment, the compositions of the invention are combined with other dietary supplement compositions. In yet other embodiments, the composition of the invention is a pharmaceutical formulation.

Depending on the intended mode of administration, the composition can be in the form of solid, semi-solid, or liquid dosage forms, such as, for example, tablets, suppositories, pills, capsules, powders, liquids, aerosols, or suspensions, preferably in unit dosage form suitable for single administration of a precise dosage. The compositions will include a therapeutically effective amount of the compound described herein or derivatives thereof in combination with a pharmaceutically acceptable carrier and, in addition, can include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the composition in which it is contained.

Throughout, treat, treating, and treatment refer to a method of reducing or delaying one or more effects or symptoms of a neurodegenerative disease or disorder. The subject can be diagnosed with disease or disorder. Treatment can also refer to a method of reducing the underlying pathology rather than just the symptoms. The effect of the administration to the subject can have the effect of but is not limited to reducing one or more symptoms of the neurodegenerative disease or disorder, a reduction in the severity of the neurological disease or injury, the complete ablation of the neurological disease or injury, or a delay in the onset or worsening of one or more symptoms. For example, a disclosed method is considered to be a treatment if there is about a 10% reduction in one or more symptoms of the disease in a subject when compared to the subject prior to treatment or when compared to a control subject or control value. Thus, the reduction can be about a 10, 20, 30, 40, 50, 60, 70, 80, 90, 100%, or any amount of reduction in between the mentioned values.

As utilized herein, by prevent, preventing, or prevention is meant to be a method of precluding, delaying, averting, obviating, forestalling, stopping, or hindering the onset, incidence, severity, or recurrence of the neurodegenerative disease or disorder. For example, the disclosed method is considered to be a prevention if there is a reduction or delay in onset, incidence, severity, or recurrence of neurodegeneration or one or more symptoms of neurodegeneration (e.g., tremor, weakness, memory loss, rigidity, spasticity, atrophy) in a subject susceptible to neurodegeneration as compared to control subjects susceptible to neurodegeneration that did not receive an agent that activates parkin. The disclosed method is also considered to be a prevention if there is a reduction or delay in onset, incidence, severity, or recurrence of neurodegeneration or one or more symptoms of neurodegeneration in a subject susceptible to neurodegeneration after receiving an agent that promotes parkin activity as compared to the subject's progression prior to receiving treatment. Thus, the reduction or delay in onset, incidence, severity, or recurrence of neurodegeneration can be about a 10, 20, 30, 40, 50, 60, 70, 80, 90, 100%, or any amount of reduction in between.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

In an embodiment, the administering comprises at least one of intravenous administration, intramuscular administration, intranasal administration, intrathecal administration, intrastriatal administration, intracranial administration, intraarterial administration, and subcutaneous administration.

In an embodiment, the ratio of Bacopa to Ashwagandha extract may range from 1:1 to 2:1 by weight, optimized to produce synergistic modulation of neuronal viability and cognitive biomarkers.

In some embodiments, the composition further comprises Amla extract (Emblica officinalis) such that the composition comprises tannins in the ranges from 10% to 30% w/V of total composition.

In some embodiments, the composition further comprises Amla extract (Emblica officinalis) such that the composition comprises tannins in the ranges from 5% to 15%.

In some embodiments, the composition further comprises Boswellia serrata extract such that the composition comprises boswellic acids range from 30% to 60%.

In some embodiment, the composition further comprises a pharmaceutically acceptable carrier.

In some embodiment, the composition further comprises a nutraceutical acceptable carrier.

In some embodiments, the composition co-administered with phosphatidylserine or B-complex vitamins enhance neuronal membrane stability.

An embodiment relates to a method for improving memory and focus on ADHD subjects by administering the composition daily for 8-12 weeks. In some embodiments, the formulation is formulated as capsules, tablets, or softgels for oral use.

In some embodiments, the formulation is formulated as powder blends for nutritional beverages.

In some embodiments, the formulation is formulated as functional foods or dietary supplements.

In an embodiment, the composition provides synergistic efficacy. This combination creates a synergistic effect, wherein Ashwagandha extract's HPA axis modulation and anti-inflammatory action create a favorable neurological environment for the potent antioxidant and cholinergic-enhancing effects of Bacopa extract to act more effectively. Combined Bacopa and Ashwagandha extracts exhibit enhanced neuroprotective and cognitive effects relative to individual administration.

In some embodiments, the composition provides dual-mode activity by simultaneous modulation of oxidative stress and neuroplasticity pathways.

In some embodiments, the composition provides stress resilience. It causes reduction in cortisol and improvement in cellular stress tolerance.

In some embodiments, the composition provides neurodegenerative protection. In some embodiments, the composition has broad therapeutic potential. The composition is effective across aging-related, degenerative, and attention-related cognitive disorders. These effects are supported by mechanistic studies and prior art analysis, indicating a novel synergistic mechanism not previously optimized or quantified.

In certain embodiments, the herbal composition is configured for use in promoting and maintaining neurological health and function. The composition may be administered for cognitive enhancement in healthy adults or elderly individuals, supporting improved memory, focus, and learning capacity. In some embodiments, the composition serves as an adjunct therapy for Alzheimer's disease and mild cognitive impairment, contributing to the reduction of oxidative stress and supporting neuronal survival. The composition may further be utilized as a supportive treatment in attention deficit hyperactivity disorder (ADHD), enhancing attentional control and cognitive processing. Additionally, the composition may aid in stress management and reduce neuroendocrine fatigue, thereby improving overall mental resilience and performance under stress. In further embodiments, the composition may function as a preventive nutraceutical for neurodegeneration and age-associated memory decline, helping to preserve neuronal integrity and delay cognitive deterioration associated with aging.

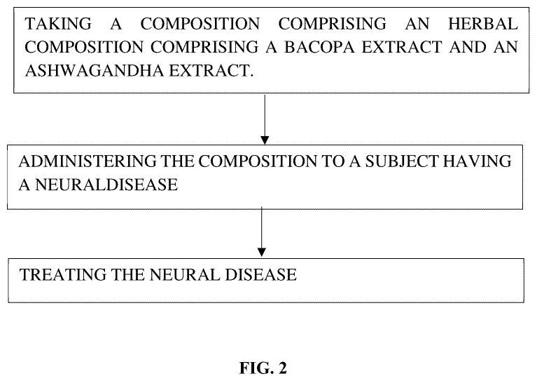

Referring to FIG. 2, it illustrates a method of treatment using the herbal composition. The method comprises taking a therapeutically effective amount of a herbal composition comprising an Ashwagandha extract and a Brahmi extract, optionally in combination with one or more active agents, vitamins, or pharmaceutically acceptable excipients. In an embodiment, the method further comprises administering the herbal composition to the subject by oral, sublingual, or enteral administration, wherein the dosage form is selected from a tablet, capsule, powder, granule, syrup, or suspension. The herbal composition may be administered as a single dose or a plurality of doses over a defined treatment period.

Upon administration, the herbal composition exerts antioxidant, anti-inflammatory, cytoprotective, and neuroprotective effects, thereby treating the neural disorder by restoring viability of neural cells, decreasing cytotoxicity, scavenging free radicals, increasing the activity of endogenous antioxidant enzymes, and preventing or reducing neuronal degeneration. The method results in an improvement or stabilization of symptoms associated with the neural disorder, including impaired cognition, memory decline, neuroinflammation, oxidative stress, impaired neurotransmission, neuronal loss, or structural or functional abnormalities in the central or peripheral nervous system.

In certain embodiments, the neural disorder treated by the method is selected from the group consisting of Alzheimer's disease, Parkinson's disease, mild cognitive impairment, age-associated cognitive decline, dementia, attention deficit hyperactivity disorder (ADHD), traumatic brain injury, neuropathic pain, migraine, anxiety-related neurological dysfunction, neurodegenerative disorders, and combinations thereof.

In an embodiment, the administering comprises at least one of intravenous administration, intramuscular administration, intranasal administration, intrathecal administration, intrastriatal administration, intracranial administration, intraarterial administration, and subcutaneous administration.

In an embodiment, the administration is in an oral dosage form.

### EXAMPLES

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Examples 1. Determination of the Antioxidant Potential of Ashwagandha and Bacopa Extract Using 2,2-Diphenyl-1-Picrylhydrazyl (DPPH Assay)

Below are provided details of the test items:

| # | Test item | Scientific Name |
|---|---|---|
| 1 | Ashwagandha | Withania somnifera |
| 2 | Brahmi | Brahmiis Bacopa |

### Principle of the Assay

DPPH (2,2-diphenyl-1-picrylhydrazyl) assay is the most widely reported preliminary method for screening of antioxidant activity of molecules. Oxidation reactions can produce free radicals, which start chain reactions that damage cells. Antioxidants terminate these chain reactions by removing free radical intermediates and inhibit other oxidation reactions by getting self-oxidized. As a result, antioxidants are often reducing agents such as thiols, ascorbic acid or polyphones. The scavenging reaction between (DPPH) and an antioxidant (H-A) can be written as:

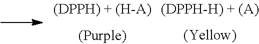

A freshly prepared DPPH solution exhibits a deep purple color with a maximum absorption at 517 nm. Antioxidants react with DPPH (purple) and get reduced to DPPH-H (yellow). This conversion decreases the absorbance and gives a measure of antioxidant potential. The degree of discoloration indicates the scavenging potential of the antioxidant compound or extract in terms of hydrogen donating ability. The free radical scavenging activity of Ashwagandha & Brahmi were evaluated spectrophotometrically by DPPH assay in vitro.

### Chemicals/Reagents/Kits

The list of chemicals and reagents used in the study are mentioned in Table 1.

TABLE 1
List of chemicals and reagents

| Name of Chemical/Reagent | Manufacturer | Batch/Lot No. |
|---|---|---|
| DPPH | Sigma Aldrich | D9132-1G |
| Methanol | Rankem | 67-56-1 |

### Preparation of Buffers and Reagents for Dpph Assay

40.4 mL of methanol was dissolved in 9.6 mL of milliQ water to make 50 ml of 80% methanol solution. 1.39 mg of DPPH was weighed and dissolved in 14.1 mL of 80% methanol. This solution was then sonicated in ultra sonicator for 45 mins till it got properly dissolved. The solution thus obtained was considered as 250 μM DPPH solution.

### Preparation of the Test Item Extract

259.51 mg of Ashwagandha was dissolved in 1.038 mL of methanol and was kept for overnight extraction at room temperature. The extract was centrifuged and considered stock solution (250 mg/ml). Stock solution was diluted using 80% Methanol to achieve final concentrations range (0.01 mg/ml to 25 mg/ml) for antioxidant potential evaluation.

273.53 mg of Brahmi was dissolved in 1.094 mL of methanol and kept for overnight extraction at room temperature. The extract was centrifuged and considered stock solution (250 mg/ml). Stock solutions were diluted using 80% Methanol to achieve final concentrations range (0.01 mg/ml to 10 mg/ml) for antioxidant potential evaluation.

### FORMULATION OF POSITIVE CONTROL (L-Ascorbic Acid)

1 mg of ascorbic acid was dissolved in 1 ml of methanol and considered as stock solution (1 mg/mL). Stock solution was further diluted using 80% methanol to achieve a final concentration range (1 μg/mL to 60 μg/mL) for antioxidant potential evaluation

### Determination of Antioxidant Potential (Procedure)

50 μl of Ashwagandha, Brahmi and L-Ascorbic acid (Positive control) at different concentrations ranging defined in section 7 and 8 were taken in 96 well micro titer plate. Additionally, a combination of Ashwagandha and Brahmi extracts at concentrations of 5 mg/ml and 1 mg/ml, respectively, was tested to evaluate the synergistic (combination) effect. 100 μl of freshly prepared 250 μM DPPH solution was added to the above plate. A blank solution of DPPH with solvent (80% methanol) was used as blank solution. The plate was then covered with aluminum foil and incubated for 30 minutes at 37 °C. The absorbance of the wells was read at 517 nm using multiwell reader.

The free radical scavenging activity of test item was evaluated at different concentrations by calculating % reduction in absorbance using the following formula:

% reduction=(Ac−As)/Ac*100,
Ac=Absorbance of Blank,
As=Absorbance of Sample
   Scavenging activity of Ashwagandha and Brahmi were calculated by its IC₅₀ value (concentration of the test solution leading to 50% decrease in absorbance compared to that of blank) using Graph Pad Prism™ software.

The free radical scavenging activity of Ashwagandha and Brahmi was evaluated spectrophotometrically using DPPH assay. The antioxidant activity is determined by the change in color from purple to yellow and subsequent fall in absorbance of the stable radical DPPH (due to decreased radical formation) at 517 nm.

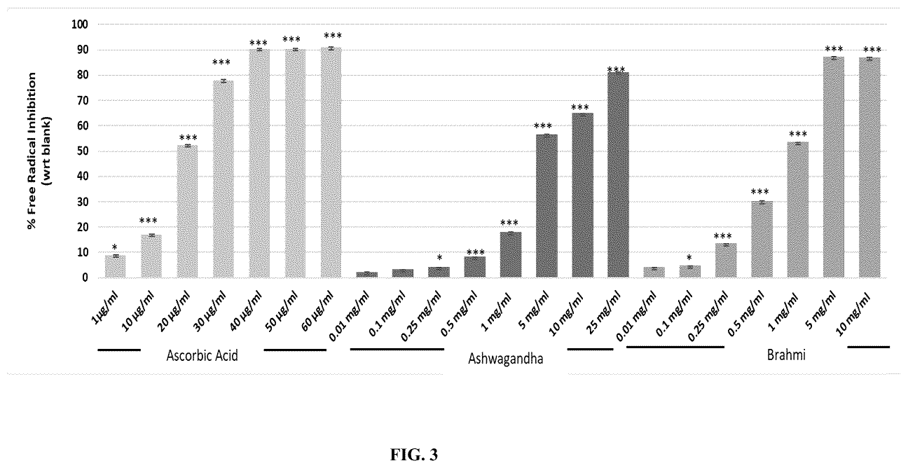

The radical scavenging potential of test items and L-ascorbic acid in terms of % reduction in absorbance and IC₅₀ values have been mentioned in Table 2 and graphically represented in FIG. 3. The IC₅₀ value of Ashwagandha, Brahmi and Ascorbic Acid was found to be 4.7 mg/ml, 1 mg/ml and 18.8 μg/ml respectively.

TABLE 2
Effect of test items on free radical scavenging activity

| | | | % | IC 50 | |
|---|---|---|---|---|---|
| Sample | Concentration | | Inhibition | value | |
| Blank | 0 | | 0 | | |
| Ascorbic Acid | 1 | μg/ml | 9 | 18.8 | μg/ml |
| | 10 | μg/ml | 17 | | |
| | 20 | μg/ml | 53 | | |
| | 30 | μg/ml | 78 | | |
| | 40 | μg/ml | 91 | | |
| | 50 | μg/ml | 91 | | |
| | 60 | μg/ml | 91 | | |
| Ashwagandha | 0.01 | mg/ml | 2 | 4.7 | mg/ml |
| | 0.1 | mg/ml | 3 | | |
| | 0.25 | mg/ml | 4 | | |
| | 0.5 | mg/ml | 8 | | |
| | 1 | mg/ml | 18 | | |
| | 5 | mg/ml | 57 | | |
| | 10 | mg/ml | 65 | | |
| | 25 | mg/ml | 81 | | |
| Brahmi extract | 0.01 | mg/ml | 4 | 1 | mg/ml |
| | 0.1 | mg/ml | 5 | | |
| | 0.25 | mg/ml | 13 | | |
| | 0.5 | mg/ml | 30 | | |
| | 1 | mg/ml | 53 | | |
| | 5 | mg/ml | 87 | | |
| | 10 | mg/ml | 87 | | |

| | | | |
|---|---|---|---|
| Ashwagandha:Brahmi | 5 mg/ml:1 mg/ml | 75 | NA |
| extract | | | |

Antioxidant potential of Ashwagandha and Brahmi were determined based on the ability to scavenge stable radical DPPH. DPPH, a nitrogen centered stable free radical is commonly used as a tool to evaluate the free radical scavenging activity of compounds.

Results obtained demonstrated the following:
Ashwagandha in the concentration range of 0.01 mg/ml-25 mg/ml demonstrated antioxidant effect by reducing DPPH radical from 2%-81% as compared to blank control with IC₅₀ value 4.7 mg/ml.
Brahmi in the concentration range of 0.01 mg/ml-10 mg/ml demonstrated antioxidant effect by reducing DPPH radical from 4%-87% as compared to blank control with IC 50 value 1 mg/ml.
Combination of Ashwagandha and Brahmi at concentration of 5 mg/ml: 1 mg/ml demonstrated enhanced antioxidant effect by reducing DPPH radical by 75% as compared to blank control.

### Example 2. Evaluation of Neuroprotective Potential of Bacopa and Ashwagandha Extracts

Below is provided details of the test items:

| # | Test item | Scientific Name |
|---|---|---|
| 1 | Brahmi | Brahmiis Bacopa |
| 2 | Ashwagandha | Withania somnifera |

### Principle of the Assay

Neuronal damage during neurodegenerative disorders is attributed to oxidative stress and neuronal toxins. The neurotoxin MPTP causes neurotoxicity through the active metabolite MPP+ (1-methyl-4-phenylpyridinium). MPP+ leads to neuronal cytotoxicity (reduced cell viability) when it is selectively taken up by dopaminergic neurons. Neurodegenerative disorders are heterogeneous groups of disorders that are characterized by the progressive degeneration of the structure and function of the central nervous system or peripheral nervous system. The common neurodegenerative diseases include Alzheimer's disease and Parkinson's disease. Neuronal damage results in oxidative injury of neurons resulting in memory loss and cognitive functions. Neuroprotection is the mechanism to defend the central nervous system (CNS) against injury due to chronic neurodegenerative disorders (e.g. Dementia, Parkinson's, Alzheimer's, Epilepsy etc.). Hence, in the present study, the effect of Brahmi and Ashwagandha extract on neuroprotection of SHSY-5Y cells against MPP+ iodide induced neuronal damage was evaluated by MTT assay.

### Chemicals/Reagents/Kits

The chemicals and reagents used in the study are listed in Table 3.

TABLE 3
List of chemicals and reagents

| S. No. | Item | Manufacture | Catalog no. | Batch/Lot No. |
|---|---|---|---|---|
| 1. | Antibiotic | Gibco | 15240-0622 | 2257209 |
| 2. | DMEM | Himedia | AL007A | 0000693247 |
| 3. | DMSO | Rankem | D0170 | R063G19 |
| 4. | FBS | Gibco | 16000-044 | 22250440P |
| 5. | MTT | Sigma | M2128-5G | MKCD8033 |
| 6. | NaCl | Rankem | S0171 | Z047B25 |
| 7. | KCl | Sigma | P9333 | BCCG6737 |
| 8. | Na₂HPO₄ | Sigma | S5136 | BCCG1754 |
| 9. | KH₂PO₄ | Sigma | P9791 | 0000375076 |
| 10. | Trypsin | Sigma | T4799 | SLCN9300 |
| 11. | EDTA | TM MEDIA | 328 | C3C8CV01 |

### Preparation of the Test Item Extract

200 mg of Ashwagandha extract was dissolved in 1 mL of DMSO to obtain a stock solution of 200 mg/mL. Stock solution was further diluted using serum free medium to prepare dilutions in the range of 3.125 μg/mL-100 μg/mL. 200 mg of Ashwagandha extract was dissolved in 1 mL of DMSO to obtain a stock solution of 200 mg/mL. Stock solution was further diluted using serum free medium to prepare dilutions in the range of 3.125 μg/mL-100 μg/mL.

### Formulation of Positive Control

9.364 mg of Selegiline was dissolved in 1 ml of DMSO and considered as stock solution of 50 mM. Stock solution was further diluted using serum free medium to prepare dilutions in the range of 0.01 μM-50 μM.

### Determination of Neuroprotective Potential of Brahmi and Ashwagandha Extract Using Shsy-5Y Cells

SHSY-5Y cells were plated in 96-well plates at density of 25000 cells/well and incubated overnight at 37 °C, 5% CO₂. After overnight incubation, cells were treated with Test Item and Positive Control at different concentrations. Additionally, a combination of Ashwagandha and Brahmi extracts, each at a concentration of 25 μg/ml, was tested to evaluate the synergistic (combination) effect. After 24 hours of incubation, neuronal damage was induced by addition of 3 mM MPP+ iodide. Cells were further incubated for overnight at 37 °C, 5% CO₂. After overnight incubation, the plates were taken out and neuroprotection was determined by MTT assay: 20 μL of 5 mg/mL of MTT 3-(4,5-dimethythiazol-2-yl)-2,5-diphenyl tetrazolium bromide solution was added to all the wells followed by additional incubation for 3 h at 37 °C. Supernatants were aspirated and 150 μL of DMSO was added to each well to dissolve formazan crystals. The absorbance of each well was be read at 540 nm using Synergy SHIM micro plate reader. The Percentage protection was calculated as:

% Neuroprotection=[(Absorbance of TI+Neuronal Damage)−(Absorbance of Neuronal Damage)/(Absorbance of Vehicle)−(Absorbance of Neuronal Damage alone)]*100

### Results

Neuroprotective potential of Ashwagandha and Brahmi Extract on SHSY-5Y cells against MPP+ iodide induced neuronal damage was determined. SHSY-5Y cells were treated with Test Items (3.125 μg/mL-100 μg/mL) and for 24 h at 37 °C in CO2 incubator. Additionally, a combination of Ashwagandha and Brahmi extracts, each at a concentration of 25 μg/mL, was tested to evaluate the synergistic neuroprotective effect. Cells were then exposed to 3 mM MPP+ iodide in the presence of test items overnight.

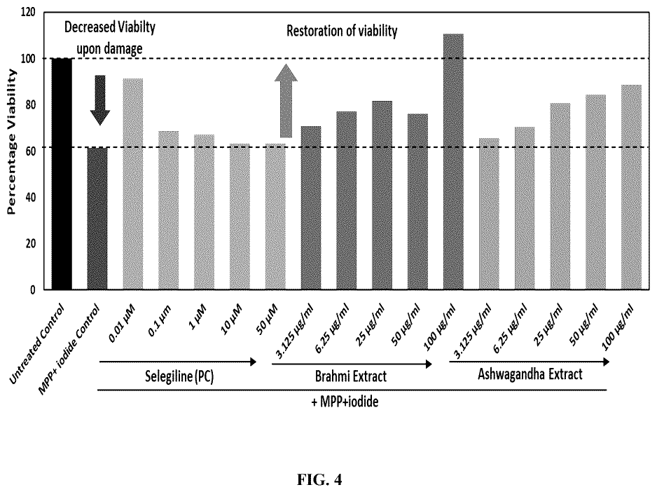

Table 4 and FIG. 4 represent the Neuroprotective potential of Brahmi and Ashwagandha Extract on SHSY-5Y cells.

Brahmi Extract in the concentration range of 3.125 μg/mL-100 μg/mL demonstrated neuroprotective potential by restoration of cell viability from 25.0%-127.3% as compared to Control (MPP+ iodide). Maximum neuroprotection of 127.3% was observed in SHSY-5Y at 100 μg/ml as compared to Control (MPP+ iodide).

Ashwagandha Extract in the concentration range of 3.125 μg/mL-100 μg/mL demonstrated neuroprotective potential by restoration of cell viability from 10.8%-70.8% as compared to Control (MPP+iodide). Maximum neuroprotection of 127.3% was observed in SHSY-5Y at 100 μg/ml as compared to Control (MPP+iodide).

The combination of Brahmi and Ashwagandha extracts at a concentration of 25 μg/mL demonstrated neuroprotective potential, restoring cell viability by 84.4% compared to the MPP+iodide-treated control. The neuroprotection of 59.6% was observed in SH-SY5Y cells compared to the MPP+iodide control.

TABLE 4
Effect of Brahmi and Ashwagandha extract on neuroprotection
on SHSY-5Y against neuronal damage

| | Sample | | | % Cytotoxicity | % Viability | % Protection |
|---|---|---|---|---|---|---|
| | | Untreated | | 0.0 | 100.0 | |
| | | Control | | | | |
| | | MPP + Iodide | | 38.6 | 61.4 | |
| | | Control (3 mM) | | | | |
| MPP + | Selegiline | 0.01 | μM | 8.5 | 91.5 | 78.0 |
| Iodide | | 0.1 | μm | 31.2 | 68.8 | 19.2 |
| (3 mM) | | 1 | μM | 32.6 | 67.4 | 15.5 |
| | | 10 | μM | 36.7 | 63.3 | 4.9 |
| | | 50 | μM | 36.8 | 63.2 | 4.5 |
| | Brahmi | 3.125 | μg/mL | 29.0 | 71.0 | 25.0 |
| | Extract | 6.25 | μg/mL | 22.7 | 77.3 | 41.3 |
| | | 25 | μg/mL | 18.2 | 81.8 | 52.9 |
| | | 50 | μg/mL | 23.8 | 76.2 | 38.3 |
| | | 100 | μg/mL | −10.5 | 110.5 | 127.3 |
| | Ashwagandha | 3.125 | μg/mL | 34.4 | 65.6 | 10.8 |
| | Extract | 6.25 | μg/mL | 29.6 | 70.4 | 23.4 |
| | | 25 | μg/mL | 19.2 | 80.8 | 50.3 |
| | | 50 | μg/mL | 15.5 | 84.5 | 59.8 |
| | | 100 | μg/mL | 11.3 | 88.7 | 70.8 |

| | | | | |
|---|---|---|---|---|
| Brahmi + | 25 μg/mL + 25 | 15.6 | 84.4 | 59.6 |
| Ashwagandha | μg/mL | | | |
| Extract | | | | |

Neuronal Damage plays a crucial role in neurological disorders such as Parkinson's Disease. MPP+ Iodide has been widely employed as neurotoxin in cells to induce neuronal damage. Cytoprotective effect against MPP+ Iodide induced cell death in neuronal cells suggest neuroprotective potential of test compounds. In the present study, effect of Brahmi and Ashwagandha Extract on Neuroprotection was studied by evaluation of restoration of viability of SHSY-5Y cells against MPP+iodide induced neuronal damage using MTT assay. Results demonstrated that Brahmi and Ashwagandha Extract led to restoration of viability of SHSY-5Y by maximum of 127.3% and 70.8% respectively as compared to MPP+iodide control cells. Additionally, the combination of Ashwagandha and Brahmi extracts at a concentration of 25 μg/mL restored cell viability by 59.6%, indicating a moderate synergistic neuroprotective effect. This indicates that Brahmi and Ashwagandha Extract may have neuroprotective potential.

The foregoing results indicate that when combined at specific sub-effective concentrations, the extracts produced synergistic enhancement of neuronal survival against MPP+-induced oxidative and mitochondrial stress. The observed synergy supports the inventive concept of co-formulated Bacopa-Ashwagandha compositions for neuroprotection and cognitive enhancement.

Accordingly, the preceding merely illustrates the principles of the disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112 (f) or 35 U.S.C. § 112 (6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112 (6) is not invoked.

It should be noted that the flowchart and the suggested time limits and parameters are meant to be exemplary, and that there could be other measures or criteria used in order to maximize safety and accuracy.

### GENERAL DEFINITIONS

Publications cited herein and the material for which they are cited are hereby specifically incorporated by reference in their entireties. A number of embodiments have been described. Nevertheless, it will be understood that various modifications may be made. Accordingly, other embodiments are within the scope of the following claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. For example, the term “about” modifies a numerical value to include ±10% of the stated value or the experimental error of measurement, whichever is greater.

Unless defined otherwise, all technical and scientific terms used herein have the same meaning as commonly understood by one of ordinary skills in the art to which this disclosure belongs. Although any methods and materials similar or equivalent to those described herein can also be used in the practice or testing of the present disclosure, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior invention. Further, the dates of publication provided may be different from the actual publication dates, which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as an antecedent basis for use of such exclusive terminology as “solely,” “only” and the like in connection with the recitation of claim elements, or use of a “negative” limitation.

As used herein, the term “comprising” is used in its open, inclusive sense; additional, unrecited elements or steps may be present.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### INCORPORATION BY REFERENCE

All references, including granted patents and patent application publications, referred herein are incorporated herein by reference in their entirety.
WO2016166565A1 titled “Use of a withania extract for the treatment of alpha synucleinopathies”;
U.S. Pat. No. 8,828,377B2 titled “Boswellia oil, its fractions and compositions for enhancing brain function”;
WO2020031209A1 titled “Method for preparing a dietary supplement and formulation thereof”;
US20070196318A1 titled “Cosmetic herbal compositions”;
U.S. Pat. No. 7,241,461B2 titled “Compositions for alleviating inflammation and oxidative stress in a mammal”; and

U.S. Pat. No. 7,923,045B2 titled “Compositions for alleviating inflammation and oxidative stress in a mammal”.

1. A composition, comprising: an herbal composition comprising a first active comprising a purified and isolated Bacopa extract and a purified and isolated Ashwagandha extract;
wherein a ratio of a concentration of the Ashwagandha extract to the Bacopa extract in the composition wherein the ratio is in a range of 2:1 to 5:1;
wherein the composition is formulated as an oral dosage form, wherein the oral dosage form is selected from the group consisting of a tablet; capsule; and caplet;
wherein the oral dosage form is sustained release particles;
wherein free radical scavenging activity of the herbal composition measured according to Formula (I) is at least 70% more compared to a blank, wherein the Formula (D) is:
% reduction=(Ac−As)/Ac*100,
Ac=Absorbance of the blank,
As=Absorbance of the herbal composition; and
wherein the blank is 250 μM 2,2-diphenyl-1-picrylhydrazyl (DPPH) solution in methanol solvent.

2-5. (canceled)

6. The composition of claim 1, wherein said composition is configured to treat a neural disorder.

7. The composition of claim 1, wherein said composition comprises a second active agent, and wherein the second active agent is selected from the group consisting of pregnenolone, galangin, vinpocetine, astaxanthin, and huperzine A.

8. The composition of claim 1, wherein the composition further comprises a vitamin.

9. The composition of claim 8, wherein the vitamin comprises Vitamin C, and wherein the amount of Vitamin C is at least 100 times less than the herbal composition.

10. The composition of claim 1, wherein the ashwagandha extract and/or the Bacopa extract is in an organic solution.

11. The composition of claim 10, wherein the organic solution is Dimethyl sulphoxide (DMSO).

12. (canceled)

13. The composition of claim 1, wherein the composition has a % DPPH inhibition more than 20 μg/ml of ascorbic acid.

14. The composition of claim 1, wherein the composition further comprises one or more excipients.

15-16. (canceled)

17. The composition of claim 16, wherein the one or more excipients selected from the group consisting of calcium carbonate; croscarmellose sodium; dicalcium phosphate; magnesium stearate; microcrystalline cellulose; modified cellulose; silica; and stearic acid.

18. The composition of claim 1, wherein the composition is configured to increase an enzyme activity level of superoxide dismutase and/or catalase.

19-20. (canceled)

21. The composition of claim 1, wherein the sustained release particles release the first active agent for 8 to 12 hours in a subject in which the composition is administered.

22. The composition of claim 1, wherein the herbal composition consists of purified and isolated Bacopa extract and a purified and isolated Ashwagandha.

23. The composition of claim 1, wherein the composition is configured to decrease plasma concentration level of reactive chemical species, when administered in an effective amount to a mammalian subject in a need thereof.
